(19) United States
(12) Patent Application Publication
(10) Pub. No.: US 2006/0135777 A1
(43) Pub. Date: Jun. 22, 2006

(54) Method for the production of d-threo-2-phenyl-2-piperidine-2-yl acetates

(75) Inventors: Huldreich Trafelet, Strengelbach (CH); Markus Wollenweber, Zofingen (CH)

Correspondence Address: HOFFMANN & BARON, LLP, 6900 JERICHO TURNPIKE, SYOSSET, NY, 11791, US

(73) Assignee: Siegfried Ltd., Zofingen (CH)

(21) Appl. No.: 10/544,791

(22) PCT Filed: Jan. 27, 2004
(86) PCT No.: PCT/IB04/00265

§ 371 Date: Feb. 23, 2006

(30) Foreign Application Priority Data
Feb. 5, 2003 (CH) 0163/03

(51) Int. Cl. C07D 211/34

(52) U.S. Cl. 546/238

(57) ABSTRACT

Disclosed is a method for producing d-threo-[R(R*,R*)]-2-phenyl-2-piperidine-2-yl acetate and the acid addition salts thereof by converting d-threo-[R(R*,R*)]-2-phenyl-2-piperidine-2-yl ethanoic acid or an acid addition salt thereof into the corresponding d-threo-[R(R*,R*)]-2-phenyl-2-piperidine-2-yl ethanoic acid halide, producing the desired d-threo-[R(R*,R*)]-2-phenyl-2-piperidine-2-yl-acetate or an acid addition salt thereof by means of esterification, and optionally recrystallizing the obtained product. The invention also relates to methods for producing pure threo-2-phenyl-2-piperidine-2-yl acetamide or the starting material for producing d-threo-[R(R*,R*)]-2-phenyl-2-piperidine-2-yl ethanoic acid.

### CROSS-REFERENCE TO RELATED APPLICATIONS:

This application is the National Stage of International Application No. PCT/IB2004/00265, which claims the benefit of Switzerland Application No.0163/03, filed Feb. 5, 2003, the contents of which are incorporated by reference herein.

### FIELD OF THE INVENTION

The present invention relates to a method for the preparation of d-threo-2-phenyl-2-piperidine-2-yl acetates and their pharmaceutically acceptable acid addition salts, particularly the preparation method of d-threo-[R(R*,R*)]-2-phenyl-2-piperidine-2-yl-methylacetate-hydrochlorides (d-threo-methyl-phenidate-hydrochloride). D-threo-2-phenyl-2-piperidine-2yl-acetates and their addition acid salts are well known. Particularly d-threo-Methylphenidate-Hydrochloride is a well known pharmaceutically effective compound.

### BACKGROUND OF THE INVENTION

D-threo-Methylphenidate and the corresponding acid addition salts as well as their preparation are known from U.S. Pat. No. 2,507,631 and from U.S. Pat. No. 2,957,880. EP-A-O 948 484 describes the accumulation of threo-Methylphenidate and corresponding addition acid salts in the same enantiomeric mixtures by using crystallization methods.

There is still a need to develop a method of preparation, particularly for d-threo-methylphenidate-Hydrochloride, so that the rate of production and the optical purity as well as the cost effectiveness of the product are improved.

### DETAILED DESCRIPTION

It has recently been discovered that, d-threo-[R(R*,R*)]-2-phenyl-2-piperidine-2-yl-acetates as well as the corresponding acid addition salts, particularly d-threo-methylphenidate-hydrochloride can be prepared from d-threo-[R(R*,R*)]-2-phenyl-2-piperidine-2-yl-acetic acid without affecting the stereo-specific properties.

In this process, d-threo-[R(R*,R*)]-2-piperidine-2-yl-acetic acid is first converted to the corresponding d-threo-[R(R*,R*)]-2-phenyl-2piperidine-2-yl-acetic acid halide, preferably to d-threo-[R(R*,R*)]-2-piperidine-2-yl acetic acid chloride and subsequently the ester is derived from this compound.

This invention, in particular, relates to a method of preparation of d-threo-[R(R*,R*)]-2-phenyl-2-piperidine-2-yl-acetates and their corresponding acid addition salts. In this method, d-threo-[R(R*,R*)]-2-phenyl-2-piperidine-2-yl-acetic acid or any one of its addition acid salts is converted into the corresponding d-threo-[R(R*,R*)]-2-phenyl-2-piperidine-2-yl-acetic acid halide, preferably into d-threo-[R(R*,R*)]-2-phenyl-2-piperidine-2-yl-acetic acid chloride, from which the desired d-threo-[R(R*,R*)]-2-phenyl-2-piperidine-2-yl-acetate or any of its acid addition salts can be prepared through esterification and the product thus obtained is recrystallized, if necessary.

Among esters (C1-4)-Alkylesters can be considered, particularly the methyl ester. Preferably d-threo-methylphenidate-hydrochloride is prepared.

Addition salts are, for example, the salts of hydrochloric acid, hydrogen bromide, tartaric acid and tartaric acid derivatives, or from other related organic acids. However,the hydrochloride is preferred.

The conditions for the halogenation of d-threo-[R(R*,R*)]-2-phenyl-2-piperidine-2-yl acetic acid to produce the corresponding halides are known from analogous reactions. D-threo-[R(R*,R*)]-2-phenyl-2-piperidine-2-yl-acetic acid chloride is preferred, which is obtained through chlorination of the corresponding d-threo-[R(R*,R*)]-2-phenyl-2-piperidine-2-yl acetic acid, especially by means of thionylchloride through the usual process. The acid chloride obtained as such is converted with absolute alcohol, preferably absolute Methanol to the desired d-threo-[R(R*,R*)]-2-phenyl-2-piperidine-2-yl-acetic acid, preferably to d-threo-methylphenidate, through splitting off hydrogen halides. The obtained product can be purified through crystallization. Thereby a product is obtained, whose enantiomaric excess (ee) for d-threo-[R(R*,R*)]-2-phenyl-2-piperidine-2-yl-acetate, shows at least 98% (ee>98%).

The halogenation reaction is carried out preferably at a temperature range of below 90° C., especially between 70° C. to 80° C., whereby after completion of halogenation and removal of the volatile components, it is immediately esterified.

The esterification reaction with alcohol can be carried out such that, the alcohol is added to the reaction mixture, after the volatile compounds have been removed from the mixture by heating in vacuum. A reaction temperature range from 35° C. to 50° C. is preferred. The remaining alcohol in the vacuum is removed afterward to facilitate the isolation of the reaction product, as this product is normally soluble in alcohol.

The present invention further relates to a method of d-threo-Methylphenidate-Hydrochloride recrystallization, in which raw d-threo-Methylphenidate-Hydrochloride on being heated in water at a temperature of 80° C. (preferably from 50° C. to 70° C.) dissolves provided the solution, if necessary, is filtered from all possible residues, thereafter cooled down to room temperature (15-25° C.) and then gaseous hydrogen-chloride of a concentration range of 12 weight % to 20 weight % is introduced, whereupon pure d-threo-Methylphenidate-Hydrochloride precipitates and can be filtered out. The optical purity normally amounts to ee>98%. It is surprising that by this recrystallization method, any existing erythro-Isomer can be removed. Organic solvents should not be used.

D-threo-[R(R*,R*)]-2-phenyl-2-piperidine-2-yl-acetic acid can be prepared by hydrolyzing d-threo-[R(R*,R*)]-2-phenyl-2-piperidine-2-yl-acetate amide with a suitable acid, preferably hydrochloric acid.

The present investigation relates further to a method of preparation of pure d-threo-[R(R*,R*)]-2-phenyl-2-piperidine-2yl-acetate amide, which can be used as per the invention, as starting material for the preparation of corresponding carboxylic acids. The application of pure d-threo-[R(R*,R*)]-2-phenyl-2-piperidine-2yl-acetate amide has a surprising advantage, in that the end product is obtained in a very pure chemical and optical form. Moreover, considerably lower amounts can be used at the stage of amide hydrolysis, which in turn, has clear commercial advantages.

As per the invention, pure, usable d-threo-[R(R*,R*)]-2-phenyl-2-piperidine-2yl-acetate amide is prepared by converting a racemic mixture of 2-phenyl-2-piperidine-2yl-acetate amide (containing d-threo-[R(R*,R*)]-2-phenyl-2-piperidine-2yl-acetate amide) with a tartaric acid derivative in a suitable solvent, preferably in isopropanol, whereby d-threo-[R(R*,R*)]-2-phenyl-2-piperidine-2yl-acetate amide-D-tartrate is obtained. This compound crystallizes out at a temperature, preferably in the range of 25-40° C. and is separated from the mixture.

The d-threo-[R(R*,R*)]-2-Phenyl-2-piperidine-2yl-acetate amide-dibenzoyl-D-tartrate obtained as such, corresponds to the following chemical formula:

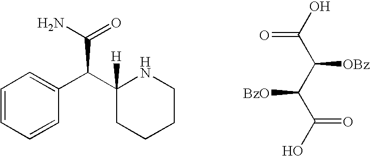

The separated d-threo-[R(R*,R*)]-2-phenyl-2-piperidine-2yl-acetate amide-dibenzoyl-D-tartrate is purified preferably through crystallization. In this method, the compound is heated till it dissolves in the solvent, preferably in Isopropanol, whereby sufficient amount of the solvent is present. It is heated preferably to a temperature of around 70° C. and then cooled down slowly to a temperature of 25° C. approximately. It crystallizes out at a temperature range of 25-37° C., preferably at 25-29° C. In this method, optical purities of ee>98% are obtained. At temperatures under 25° C., the erythro-isomer crystallizes out.

D-threo-[R(R*,R*)]-2-phenyl-2-piperidine-2yl-acetate amide can then be extracted from d-threo-[R(R*,R*)]-2-phenyl-2-piperidine-2yl-acetate amide-dibenzoyl-D-tartrate by means of alkali treatment, preferably with sodium hydroxide. Thereby d-threo-[R(R*,R*)]-2-phenyl-2-piperidine-2yl-acetate amide-dibenzoyl-D-tartrate is treated with preferably a 30% aqueous solution of sodium hydroxide at room temperature i.e at 15-25° C. and subsequently cooled to a temperature, preferably in the range of 2-10° C., whereupon d-threo-[R(R*,R*)]-2-phenyl-2-piperidine-2yl-acetate amide crystallizes out and can be filtered out.

In this regard, the present invention deals with the method of preparation of pure d-threo-[R(R*,R*)]-2-phenyl-2-piperidine-2yl-acetate amide, which is characterized by (i) a racemic mixture of 2-phenyl-2-piperidine-2yl-acetate amide converted with (+)-O,O-Dibenzoyl-D-Tartaric acid in a suitable solution, preferably in Isopropanol and the formed d-threo-[R(R*,R*)]-2-phenyl-2-piperidine-2yl-acetate-amide-dibenzoyl-D-tartrate separates from the mixture and if necessary, purified through crystallization and subsequently (ii) d-threo-[R(R*,R*)]-2-phenyl-2-piperidine-2yl-acetate-amide-dibenzoyl-D-tartrate is treated with alkali, whereby d-threo-[R(R*,R*)]-2-phenyl-2-piperidine-2yl-acetate-amide is obtained.

The racemic mixture of 2-phenyl-2-piperidine-2yl-acetate-amide containing d-threo-[R(R*,R*)]-2-phenyl-2-piperidine-2yl-acetate-amide is prepared preferably by treating 2-phenyl-2-piperidine-2yl-acetate-amide with a base, particularly aqueous sodium hydroxide, whereby a diastereomeric mixture is formed. This mixture contains a diastereomeric excess of around 85% of 2-phenyl-2-piperidine-2yl-acetate-amide. So, for instance, treatment with a 20% aqueous sodium hydroxide solution heated to about 100° C. for approximately 60 minutes, produces a mixture, which contains the threo-compound at a concentration of approximately 85%.

According to the following description, the present invention deals also with the method of preparation of d-threo-methylphenidate-hydrochloride, whose constituent steps are as follows: (1.) 2-phenyl-2-piperidine-2yl-acetate-amide is treated with a base, whereby a threo-form enriched racemic mixture is formed, (2.) the racemic mixture of 2-phenyl-2-piperidine-2yl-acetate-amide with (+)-O,O-dibenzoyl-D-tartaric acid in a suitable solvent is converted and the formed d-threo-[R(R*,R*)]-2-phenyl-2-piperidine-2yl-acetate amide-dibenzoyl-D-tartrate separated from the mixture and if necessary, purified through crystallization, subsequently d-threo-[R(R*,R*)]-2-phenyl-2-piperidine-2yl-acetateamide-dibenzoyl-D-tartrate treated with alkali and the d-threo-[R(R*,R*)]-2-phenyl-2-piperidine-2yl-acetate amide is isolated, (3.) the d-threo-[R(R*,R*)]-2-phenyl-2-piperidine-2yl-acetate amide is hydrolyzed with a suitable acid, whereby d-threo-[R(R*,R*)]-2-phenyl-2-piperidine-2yl-acetic acid can be obtained, (4.) the obtained d-threo-[R(R*,R*)]-2-phenyl-2-piperidine-2yl-acetic acid is transformed into the corresponding d-threo-[R(R*,R*)]-2-phenyl-2-piperidine-2yl-acetic-chloride and subsequently from this, the desired d-threo-methylphenidate-hydrochloride is prepared through esterification with methanol and (5.) the obtained product, if necessary, recrystallized from hydrochloric acid. The following examples elucidate the invention.

### EXAMPLE 1

### Preparation of racemic threo-2-Phenyl-2-piperidine-2yl-acetate-amide

91.6 g of 30% aqueous sodium hydroxide is diluted with 53g water in a 250 ml round bottom flask. After that, a racemic mixture of erythro/threo-2-phenyl-2-piperidine-2yl-acetate amide is added while stirring at room temperature and stirred further for 5-10 minutes till a uniform suspension is obtained. After that, it is heated slowly to 101-103° C. and this temperature is held for a further 5 hours. The temperature should not exceed 108° C. The obtained suspension is subsequently cooled down rapidly to 20±5° C. and stirred overnight. The suspension is centrifuged and the centrifugate obtained is washed carefully with water and dried in vacuum at a pressure <200 mbar and temperature of 55±5° C. A yield of 29.7 g (90%) is obtained with a threo-content of de˜85% (determined by HPLC).

### EXAMPLE 2

### Preparation of d-threo-[R(R*,R*)]-2-phenyl-2-piperidine-2yl-acetate-amide-dibenzoyl-D-tartrate

333.1 g Isopropanol as well as 16.1 g of the previously prepared (Example 1) racemic mixture containing threo-2-phenyl-2-piperidine-2yl-acetate-amide (de˜85%) and 26.5 g of dibenzoyl-D-tartaric acid are taken in a 500 ml round bottom flask at room temperature and stirred till a uniform suspension is obtained. After that, the suspension is heated to 55±5° C. and kept at this temperature for an hour and then slowly cooled down to 37° C. The temperature of 37° C. is maintained for another 90 minutes, whereupon it is further cooled down to 27° C. This temperature of 27° C. is maintained now for approximately 4 hours till all d-threo-[R(R*,R*)]-2-phenyl-2-piperidine-2yl-acetate-amide-dibenzoyl-D-tartrate crystallizes out. The temperature should not fall below 25° C. The obtained suspension is filtered and the filtercake is washed with Isopropanol at a temperature of 20-25° C. After that the moist filter cake is dried overnight in vacuum at 45-55° C. and a pressure of <200 mbar. A yield of d-threo-[R(R*,R*)]-2-phenyl-2-piperidine-2yl-acetate-amide-dibenzoyl-D-tartrate of 88.0% is calculated from d-threo-[R(R*,R*)]-2-phenyl-2-piperidine-2yl-acetate amide, in an purity of 99.5% ( determined by HPLC).

### EXAMPLE 3

### Preparation of d-threo-[R(R*,R*)]-2-phenyl-2-piperidine-2yl-acetate amide

27.9 g of water and 12.8 g of a 30% aqueous solution of sodium hydroxide are taken in a round bottom flask of 100 ml. While stirring, 17.26 g of the prepared d-threo-[R(R*,R*)]-2-phenyl-2-piperidine-2yl-acetate-amide-dibenzoyl-D-tartrate (in Example 2) is added slowly for approximately 60 minutes at a temperature in the range of 15-25° C., whereby d-threo-[R(R*,R*)]-2-phenyl-2-piperidine-2yl-acetate amide is released in an exothermic reaction. The obtained suspension is now cooled down to 4-10° C. under constant stirring and kept at this temperature for approximately 60 minutes. The suspension is then centrifuged at 4-10° C. The obtained crystals are washed at first with diluted NaOH and then with water and subsequently dried overnight in vacuum at 45-55° C. The yield of pure d-threo-[R(R*,R*)]-2-phenyl-2-piperidine-2yl-acetate amide (ee˜98%) amounts to approximately 80%.

### EXAMPLE 4

### Preparation of d-threo-[R(R*,R*)]-2-phenyl-2-piperidine-2yl-acetic acid hydrochloride

6.36 g of water and 17.34 g of 32% aqueous solution of hydrogen chloride (hydrochloric acid) are taken in a 100 ml round bottom flask. While stirring, 5.14 g of the prepared (Example 3) d-threo-[R(R*,R*)]-2-phenyl-2-piperidine-2yl-acetate amide is added at room temperature, whereby a mild exothermic reaction occurs. The obtained mixture is now heated to 100-107° C. in about an hour, under constant stirring. It is kept at this temperature for approximately 5 hours, under constant stirring. The mixture is then cooled down to a temperature range of −3° C. to 1° C. in less than an hour, whereupon the suspension is centrifuged. The obtained crystals are washed with water and subsequently dried overnight in vacuum at 45-55° C. The yield for d-threo-[R(R*,R*)]-2-phenyl-2-piperidine-2yl-acetic acid hydrochloride amounts to 4.7 g with a purity (ee) of 95% (determined by HPLC).

### EXAMPLE 5

### Preparation of d-threo-methylphenidate-hydrochloride

a) 26.0 g of Toluol, 0.02 g of N,N-dimethylformamide and 2.6 g of prepared d-threo-[R(R*,R*)]-2-phenyl-2-piperidine-2yl-acetic acid hydrochloride (as per Example 4) are taken in a 250 ml round bottom flask. After that 3.6 g of thionylchloride are added for 15 minutes at room temperature and under stirring. Subsequently the mixture is heated to 82° C. within 30 minutes and kept at this temperature for 2 hours. Thereby hydrogen chloride (HCl) and sulphurdioxide (SO₂) evolve, which are then absorbed in a NaOH laden absorber. The reaction mixture is then cooled down to 45-55° C. and analyzed by HPLC. The reaction is carried out till the concentration of acid chlorides amount to 95% at least. The reaction mixture is then set under vacuum (90-130 mbar) at a temperature of 37-53° C., in order to remove all volatile reaction components. This is achieved in an hour approximately.

b) The reaction mixture obtained in paragraph a) is now cooled down to a temperature in the range of 37-43° C. and slowly under stirring is added to 6.2 g of methanol. This causes an exothermic reaction, which is required to cool the reaction mixture, so that it is kept at a temperature of 37-43° C. After this addition, the reaction mixture is still kept for 2 hours in a temperature range of 37-50° C. and stirred. The reaction mixture is then analyzed by HPLC and the reaction carried out till the concentration of d-threo-Methylphenidate amounts to at least 95%. The reaction mixture is now set at a temperature in the range of 25-45° C. and under vacuum (90-130 mbar), so that all volatile reaction components are removed. This is achieved in an hour approximately. The mixture is then cooled down slowly to 15-20° C., whereupon the suspension is centrifuged. The obtained crystals are washed with toluol and subsequently dried overnight at 45-55° C., in vacuum. The yield for d-threo-methylphenidate-hydrochloride (raw) amounts to 2.5 g with a purity (ee) of 99% (determined by HPLC).

### EXAMPLE 6

### Purification of raw d-threo-methylphenidate-hydrochloride

6.5 g of deionised water as well as 2.42 g of prepared (Example 5) d-threo-methylphenidate-hydrochloride (raw) and 0.05 g of bleached charcoal are taken in a 50ml round bottom flask and heated to 60-70° C. in 30 minutes, while stirring and then kept at this temperature for further 30 minutes. Then it is filtered and rinsed at 60° C. with deionised water at 60° C. The clear solution obtained is cooled down to a temperature range of 15-25° C., whereby anhydrous hydrogen chloride gas is passed into the solution till a concentration of about 18 weight % is reached. During this, the temperature of the solution should not exceed 35° C. The obtained suspension is cooled down to 0-6° C. and stirred for 90 minutes at this temperature. After that, the suspension is centrifuged at a a temperature range of 0-5° C. The crystals obtained, are washed with cold isopropanol and subsequently dried overnight in vacuum at 45-55° C. The yield for pure d-threo-methylphenidate-hydrochloride (with respect to the raw starting product) amounts to 73% at a purity (ee) of >99.1% (determined by HPLC).

1. Method of preparing d-threo-[R(R*,R*)]2-phenyl-2-piperidine-2-yl-acetates and their acid addition salts compromising the steps of converting d-threo-[R(R*,R*)]2-phenyl-2-piperidine-2-yl-acetic acid or an acid addition salt into the corresponding d-threo-[R(R*,R*)]2-phenyl-2-piperidine-2-yl-acetic acid halide and preparing the d-threo-[R(R*,R*)]2-phenyl-2-piperidine-2-yl-acetate or an acid addition salt through esterification and optionally crystallizing the obtained product.

2. The method, according to claim 1, wherein, said d-threo-[R(R*,R*)]2-phenyl-2-piperidine-2-yl-acetic acid halide is d-threo-[R(R*,R*)]2-phenyl-2-piperidine-2-yl-acetic acid chloride.

3. The method, according to claim 1, wherein said d-threo-[R(R*,R*)]2-phenyl-2-piperidine-2-yl-acetate is a (C₁₋₄)-alkylester.

4. The method, according to claim 1 or 2, wherein the said d-threo-[R(R*,R*)]2-Phenyl-2-piperidine-2-yl-acetic acid hydrochloride is chlorinated with thionylchloride in an inert solvent, and in the presence of a catalytically active aprotic solvent, and, that the obtained acid chloride on reaction with absolute alcohol, is transformed into the d-threo-[R(R*,R*)]2-phenyl-2-piperidine-2-yl-acetate by splitting off hydrogen halides.

5. The method, according to claim 4, wherein the halogenation is carried out at a temperature below 90° C.

6. A purification method of d-threo-methylphenidate-hydrochloride wherein d-threo-methylphenidate-hydrochloride is crystallized out, by dissolving it in water heated at a temperature of 80° C., cooling down said solution to room temperature and introducing gaseous hydrogen chloride of at the concentration in the range of 12 weight % to 20 weight %, and filtering out the obtained pure d-threo-methylphenidate-hydrochloride as a precipitate.

7. The method, according to claim 1, wherein the d-threo-[R(R*,R*)]2-phenyl-2-piperidine-2-yl-acetic acid is prepared from d-threo-[R(R*,R*)]2-phenyl-2-piperidine-2-yl-acetate amide through hydrolysis with a suitable acid.

8. Method of preparation of pure d-threo-[R(R*,R*)]2-phenyl-2-piperidine-2-yl-acetate-amide or the starting material for the preparation of corresponding carboxylic acid, as in claim 7, wherein a racemic mixture of 2-phenyl-2-piperidine-2-yl-acetate amide is converted at first, with (+)-O,O-dibenzoyl-D-tartaric acid in a suitable solvent, preferably in isopropanol, said d-threo-[R(R*,R*)]2-Phenyl-2-piperidine-2-yl-acetate-amide-dibenzoyl-D-tartrate thus formed crystallizes at a temperature between 25-40° C. and is optionally purified, through recrystallization and subsequently through alkali treatment, and is converted into d-threo-[R(R*,R*)]2-Phenyl-2-piperidine-2-yl-acetate-amide.

9. The method according to claim 8, wherein the recrystallization of the separated d-threo-[R(R*,R*)]2-phenyl-2-piperidine-2-yl-acetate amide-dibenzoyl-D-tartrate is caused by heating up to 70° C., until complete dissolution of the compound in the solvent, and subsequently cooling down slowly to 25° C., whereby d-threo-[R(R*,R*)]2-phenyl-2-piperidine-2-yl-acetate-amide-dibenzoyl-D-tartrate crystallizes out.

10. The method as in claim 8, wherein d-threo-[R(R*,R*)]2-phenyl-2-piperidine-2-yl-acetate-amide is treated with a 30% aqueous solution of sodium hydroxide at room temperature and subsequently cooled down to 2-10° C., so that d-threo-[R(R*,R*)]2-phenyl-2-piperidine-2-yl-acetateamide crystallizes out.

11. Method of preparation of threo-2-phenyl-2-piperidine-2-yl-acetate amide or the starting product for the method, as per claim 8, wherein the said method comprises the treatment of a erythro/threo-mixture of 2-phenyl-2-piperidine-2-yl-acetate amide with a base.

12. The method, according to claim 11 wherein the erythro/threo 2-phenyl-2-piperidine-2-yl-acetate amide on being heated with a 20% aqueous sodium hydroxide solution to a temperature of 100° C. for approximately 60 minutes, produces a mixture containing approximately 85% of the threo-compound.

13. Method of preparing d-threo-methylphenidate-hydrochloride comprising (1.) reacting erythro/threo 2-phenyl-2-piperidine-2-yl-acetate amide with a base, to produce a threo enriched mixture, (2.) converting the racemic mixture of 2-phenyl-2-piperidine-2-yl-acetate amide, with(+)-O,O-dibenzoyl-D-tartaric acid in a suitable solvent, separately the formed d-threo-[R(R*,R*)]2-phenyl-2-piperidine-2-yl-acetate amide-dibenzoyl-D-tartrate from the mixture and optionally purifying through crystallization, subsequently treating the d-threo-[R(R*,R*)]2-phenyl-2-piperidine-2-yl-acetate-amide-dibenzoyl-D-tartrate is with alkali and isolating the d-threo-[R(R*,R*)]2-phenyl-2-piperidine-2-yl-acetate-amide, (3.) hydrolyzing the d-threo-[R(R*,R*)]2-phenyl-2-piperidine-2-yl-acetate amide, according to claim 7 with a suitable acid, whereby d-threo-[R(R*,R*)]2-phenyl-2-piperidine-2-yl-acetic acid is obtained, (4.) converting the obtained d-threo-[R(R*,R*)]2-phenyl-2-piperidine-2-yl-acetic acid is into the corresponding d-threo-[R(R*,R*)]2-phenyl-2-piperidine-2-yl-chlorideacetate, and subsequently preparing, d-threo-methylphenidate-hydrochloride through esterification with methanol and (5.) recrystallizing the obtained product from hydrochloric acid.
